SEQUENCE LISTING (2 sequences)

SEQ ID NO: 1 - RNA, 23 nt, synthetic construct
  source 1..23: mol_type = other RNA
taatactgccgggtaatgatgga

SEQ ID NO: 2 - protein, 5 aa, synthetic construct
KGDGG

(19) United States
(12) Patent Application Publication
(10) Pub. No.: US 2024/0182897 A1
(43) Pub. Date: Jun. 6, 2024

(54) OLIGONUCLEOTIDE THERAPEUTICS AND APPLICATION THEREOF

(71) Applicant: Vacino Biotech Co., Ltd., Taipei (TW)

(72) Inventors: Jia-Ming CHANG, Taipei (TW); Chiung-Wen Liou, Taipei (TW)

(21) Appl. No.: 18/502,329

(22) Filed: Nov. 6, 2023

Related U.S. Application Data
(60) Provisional application No. 63/385,258, filed on Nov. 29, 2022.

(51) Int. Cl. C12N 15/113; A61K 31/131; A61K 47/10; A61P 29/00

(52) U.S. Cl. CPC C12N 15/113; A61K 31/131; A61K 47/10; A61P 29/00; C12N 2310/141

(57) ABSTRACT

The present invention relates to oligonucleotide therapeutics. The oligonucleotide therapeutics have at least an oligonucleotide conjugated to a dodecylamine at the 5′ end of the oligonucleotide. The oligonucleotide therapeutics may further contain a polyethylene glycol (PEG) conjugated to the dodecylamine at the amino terminus of the dodecylamine, and may further contain a peptide linker disposed between the dodecylamine and the PEG.

### CROSS-REFERENCE TO RELATED APPLICATIONS

This application claims priority to and the benefit of U.S. Provisional Application No. 63/385,258, filed on Nov. 29, 2022, the disclosure of which is incorporated by reference in its entirety.

### SUBMISSION OF SEQUENCE LISTING AS ASCII TEXT FILE

This application includes an electronically submitted sequence listing in XML format. The XML file contains a sequence listing entitled “P23-0224US_Sequence_Listing.xml” which was created on Nov. 2, 2023 and is 2,875 bytes in size. The sequence listing contained in this XML file is part of the specification and is hereby incorporated by reference herein in its entirety.

### BACKGROUND OF THE INVENTION

### 1. FIELD OF THE INVENTION

The present invention relates to an oligonucleotide therapeutic, especially to an oligonucleotide conjugated to a dodecylamine. The oligonucleotide therapeutic may further comprises a polyethylene glycol (PEG) conjugated to the dodecylamine, and may even further comprises a peptide linker disposed between the dodecylamine and the PEG.

### 2. DESCRIPTION OF THE PRIOR ART

According to the World Health Organization (WHO) in 2023, more than 55 million people worldwide suffer from dementia, and there are about 10 million new cases every year. Alzheimer's disease (AD) is the most common neurodegenerative disease and the major cause of dementia. The disease is caused by the accumulation of β-amyloid (AB), abnormal phosphorylation of Tau protein, and excessive inflammation of nerves. Most studies suggest that β-amyloid protein causes plaque accumulation, which further causes neurotoxicity in the brain, and the hyperphosphorylation of tau protein forms neurofibrillary tangles (NFT), which causes irreversible neuron cell death (Bloom, 2014).

To date, recent researches point out and suggest that neuroinflammation is an initial cause of Alzheimer's disease (Kinney et al., 2018). This phenomena of neuroinflammation enhanced by Aβ accumulation stimulates microglia to release highly neurotoxic inflammatory factors and promotes the occurrence of inflammatory reactions in the brain (Lueg et al., 2015; Hansen et al., 2018). Therefore, it is extremely important to suppress, attenuate inflammation, especially neuroinflammation, for preventing neurodegenerative diseases, such as dementia and Alzheimer's disease.

### SUMMARY OF THE INVENTION

The present invention is based, at least in part, on the discovery that a dodecylamine modified microRNA 200c-3p (SEQ ID NO: 1) (designated as C12-miR) surprisingly increases PD-L1 expression of cells, which is completely opposite to the downregulating effects of microRNA 200c-3p mimic or microRNA 200c-3p expression vector on PD-L1 expression of cells as previously reported (Anastasiadou et al., 2021; Zhang et al., 2023). Pegylation of C12-miR and addition of the peptide linker (SEQ ID NO: 2) between the PEG and C12-miR further induce more PD-L1 expression of cells. Based on the PD-1/PD-L1 axis, the dodecylamine modified microRNA 200c-3p (SEQ ID NO: 1) (C12-miR), the pegylated C12-miRs, and the pegylated peptide-linked C12-miRs disclosed in the present invention can be used as oligonucleotide therapeutics to attenuate, suppress inflammatory responses, especially neuroinflammation.

Therefore, in some embodiments, the present invention provides oligonucleotide therapeutics comprising at least an oligonucleotide conjugated to a dodecylamine at the 5′ end of the oligonucleotide. In some other embodiments, the oligonucleotide therapeutics may further comprise a PEG conjugated to the dodecylamine at the amino terminus of the dodecylamine. In some other embodiments, the oligonucleotide therapeutics may further comprise a peptide linker disposed between the dodecylamine and the PEG.

The present invention also provides application or use of the oligonucleotide therapeutics. In some embodiments, the oligonucleotide therapeutics disclosed in the present invention can be used to increase the expression of PD-L1 of a cell. In some other embodiments, the oligonucleotide therapeutics disclosed in the present invention can be used to increase the expression of PD-L1 in a subject. In some other embodiments, the oligonucleotide therapeutics disclosed in the present invention can be used to prevent, attenuate, suppress, or treat inflammation in a subject.

Those skilled in the art will recognize, or be able to ascertain using no more than routine experimentation, many equivalents to the specific embodiments of the invention described herein. Such equivalents are intended to be encompassed by the following embodiments.

Embodiment 1. An oligonucleotide therapeutic, comprising an oligonucleotide and a dodecylamine, wherein a first carbon of the dodecylamine is conjugated to the oligonucleotide at a 5′ end of the oligonucleotide, and an amino group of the dodecylamine is located at a twelfth carbon of the dodecylamine.

Embodiment 2. The oligonucleotide therapeutic of Embodiment 1, wherein the oligonucleotide is a microRNA.

Embodiment 3. The oligonucleotide therapeutic of Embodiment 1 or 2, wherein the oligonucleotide consists of a sequence of SEQ ID NO: 1.

Embodiment 4. The oligonucleotide therapeutic of any one of Embodiments 1 to 3, further comprising a polyethylene glycol (PEG) conjugated to the dodecylamine at an amino terminus of the dodecylamine.

Embodiment 5. The oligonucleotide therapeutic of Embodiment 4, wherein the PEG is selected from the group consisting of PEG 500, PEG 1000, and PEG 2000.

Embodiment 6. The oligonucleotide therapeutic of any one of Embodiments 1 to 3, further comprising a peptide linker conjugated to the dodecylamine at an amino terminus of the dodecylamine and a PEG conjugated to the peptide linker at an amino terminus of the peptide linker.

Embodiment 7. The oligonucleotide therapeutic of Embodiments 6, wherein the peptide linker consists of a sequence of SEQ ID NO: 2.

Embodiment 8. The oligonucleotide therapeutic of Embodiments 6 or 7, wherein the PEG is selected from the group consisting of PEG 500, PEG 1000, and PEG 2000.

Embodiment 9. The oligonucleotide therapeutic of any one of Embodiments 1 to 8, wherein the oligonucleotide therapeutic is selected from the group consisting of
an oligonucleotide therapeutic consisting of an oligonucleotide and a dodecylamine, wherein a first carbon of the dodecylamine is conjugated to the oligonucleotide at a 5′ end of the oligonucleotide, and an amino group of the dodecylamine is located at a twelfth carbon of the dodecylamine;
an oligonucleotide therapeutic consisting of an oligonucleotide, a dodecylamine, and a PEG, wherein a first carbon of the dodecylamine is conjugated to the oligonucleotide at a 5′ end of the oligonucleotide, an amino group of the dodecylamine is located at a twelfth carbon of the dodecylamine, and the PEG is conjugated to the dodecylamine at an amino terminus of the dodecylamine; and
an oligonucleotide therapeutic consisting of an oligonucleotide, a dodecylamine, a peptide linker, and a PEG, wherein a first carbon of the dodecylamine is conjugated to the oligonucleotide at a 5′ end of the oligonucleotide, an amino group of the dodecylamine is located at a twelfth carbon of the dodecylamine, the peptide linker is conjugated to the dodecylamine at an amino terminus of the dodecylamine amino terminus of the dodecylamine, and the PEG is conjugated to the peptide linker at an amino terminus of the peptide linker.

Embodiment 10. The oligonucleotide therapeutic of any one of Embodiments 1 to 9, wherein the oligonucleotide therapeutic consists of an oligonucleotide and a dodecylamine, wherein a first carbon of the dodecylamine is conjugated to the oligonucleotide at a 5′ end of the oligonucleotide, and an amino group of the dodecylamine is located at a twelfth carbon of the dodecylamine.

Embodiment 11. The oligonucleotide therapeutic of any one of Embodiments 1 to 9, wherein the oligonucleotide therapeutic consists of an oligonucleotide, a dodecylamine, and a PEG, wherein a first carbon of the dodecylamine is conjugated to the oligonucleotide at a 5′ end of the oligonucleotide, an amino group of the dodecylamine is located at a twelfth carbon of the dodecylamine, and the PEG is conjugated to the dodecylamine at an amino terminus of the dodecylamine.

Embodiment 12. The oligonucleotide therapeutic of any one of Embodiments 1 to 9, wherein the oligonucleotide therapeutic consists of an oligonucleotide, a dodecylamine, a peptide linker, and a PEG, wherein a first carbon of the dodecylamine is conjugated to the oligonucleotide at a 5′ end of the oligonucleotide, an amino group of the dodecylamine is located at a twelfth carbon of the dodecylamine, the peptide linker is conjugated to the dodecylamine at an amino terminus of the dodecylamine amino terminus of the dodecylamine, and the PEG is conjugated to the peptide linker at an amino terminus of the peptide linker.

Embodiment 13. The oligonucleotide therapeutic of any one of Embodiments 9 to 12, wherein the oligonucleotide is a microRNA.

Embodiment 14. The oligonucleotide therapeutic of any one of Embodiments 9 to 13, wherein the oligonucleotide consists of a sequence of SEQ ID NO: 1.

Embodiment 15. The oligonucleotide therapeutic of any one of Embodiments 9 to 14, wherein the PEG is selected from the group consisting of PEG 500, PEG 1000, and PEG 2000.

Embodiment 16. The oligonucleotide therapeutic of any one of Embodiments 9 to 15, wherein the peptide linker consists of a sequence of SEQ ID NO: 2.

Embodiment 17. A composition, comprising at least one of the oligonucleotide therapeutics of any one of Embodiments 1 to 16 and a pharmaceutically acceptable carrier or excipient.

Embodiment 18. A method of increasing the expression of PD-L1 of a cell, comprising contacting the cell with the oligonucleotide therapeutic of any one of Embodiments 1 to 16 or the composition of Embodiment 17.

Embodiment 19. A method of increasing the expression of PD-L1 in a subject, comprising administering to the subject the oligonucleotide therapeutic of any one of

Embodiments 1 to 16 or the composition of Embodiment 17.

Embodiment 20. A method of preventing, attenuating, suppressing, or treating inflammation in a subject, comprising administering to the subject a pharmaceutically effective amount of the oligonucleotide therapeutic of any one of Embodiments 1 to 16 or the composition of Embodiment 17.

Embodiment 21. The method of Embodiment 20, wherein the inflammation in a subject is a neuroinflammation.

Embodiment 22. The oligonucleotide therapeutic of any one of Embodiments 1 to 16 or the composition of Embodiment 17 for use in increasing the expression of PD-L1 of a cell.

Embodiment 23. The oligonucleotide therapeutic of any one of Embodiments 1 to 16 or the composition of Embodiment 17 for use in increasing the expression of PD-L1 in a subject.

Embodiment 24. The oligonucleotide therapeutic of any one of Embodiments 1 to 16 or the composition of Embodiment 17 for use in preventing, attenuating, suppressing, or treating inflammation in a subject.

Embodiment 25. The use of Embodiment 24, wherein the inflammation in a subject is a neuroinflammation.

Embodiment 26. Use of the oligonucleotide therapeutic of any one of Embodiments 1 to 16 or the composition of Embodiment 17 for the manufacture of a medicament for increasing the expression of PD-L1 of a cell.

Embodiment 27. Use of the oligonucleotide therapeutic of any one of Embodiments 1 to 16 or the composition of Embodiment 17 for the manufacture of a medicament for increasing the expression of PD-L1 in a subject.

Embodiment 28. Use of the oligonucleotide therapeutic of any one of

Embodiments 1 to 16 or the composition of Embodiment 17 for the manufacture of a medicament for preventing, attenuating, suppressing, or treating inflammation in a subject.

Embodiment 29. The use of Embodiment 28, wherein the inflammation in a subject is a neuroinflammation.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows the schematic diagrams of the design of the oligonucleotide therapeutics disclosed in the present invention. miR, C12, and Linker represent microRNA, dodecylamine, and peptide linker (SEQ ID NO: 2), respectively.

FIG. 2 shows characterization of the oligonucleotide therapeutics prepared in Example 1 by electrophoresis in a 15% (v/v) acrylamide gel containing 8 M urea followed by staining with 0.2% (w/v) methylene blue. Arrows indicate the locations of the sizes (7 kDa and 8 kDa) on the gel.

FIG. 3 shows the amounts of PD-L1 expression of SH-SY5Y human neuroblastoma cells (ATCC® CRL-2266) treated with phosphate-buffered saline (PBS) (as blank control), 2 μM C12-miR, 2 μM P.5-C12-miR, or 2 μM P.5-L-C12-miR for 48 hours in Example 2. PD-L1 expression was analyzed by anti-human PD-L1 surface antibodies with a flow cytometer. Results are presented as mean with error bars representing standard error and statistical significance calculated with Student's t-test. *p<0.05, **p<0.01, ***p<0.001, compared with the blank control. # p<0.05, ### p<0.001, compared with C12-miR. †p<0.05, compared with P.5-C12-miR.

FIG. 4 shows the effects of C12-miR and P.5-C12-miR with different concentrations (0.5 μM, 2 μM, and 4 μM) on PD-L1 expression of SH-SY5Y human neuroblastoma cells (ATCC® CRL-2266) in Example 3. PD-L1 expression was analyzed by anti-human PD-L1 surface antibodies with a flow cytometer. Results are presented as mean with error bars representing standard error and statistical significance calculated with Student's 1-test. **p<0.01, ***p<0.001, compared with the blank control (0 μM). # p<0.05, ## p<0.01, compared with C12-miR.

FIG. 5 shows the effects of different sizes of PEG conjugated to C12-miR on PD-L1 expression of SH-SY5Y human neuroblastoma cells (ATCC® CRL-2266) in Example 4. PD-L1 expression was analyzed by anti-human PD-L1 surface antibodies with a flow cytometer. Results are presented as mean with error bars representing standard error and statistical significance calculated with Student's t-test. * p<0.05, compared with the positive control (C12-miR).

FIG. 6 shows size reduction of the pegylated peptide-linked C12-miR (P.5-L-C12-miR) subjected to enzyme cleavage by lysosomal enzyme Cathepsin D in Example 5. P.5-L-C12-miR (control) and P.5-L-C12-miR treated with lysosomal cathepsin D were analyzed by electrophoresis in a 15% (v/v) acrylamide gel containing 8 M urea followed by staining with 0.2% (w/v) methylene blue. Bars indicate the size of each band.

### DETAILED DESCRIPTION OF THE PREFERRED EMBODIMENT

The present invention is based, at least in part, on the discovery that a dodecylamine modified microRNA 200c-3p (SEQ ID NO: 1) (C12-miR) surprisingly increases PD-L1 expression of cells; in addition, pegylation of C12-miR and addition of the peptide linker (SEQ ID NO: 2) between the PEG and C12-miR further induce more PD-L1 expression of cells.

Therefore, the present invention provides oligonucleotide therapeutics comprising at least an oligonucleotide conjugated to a dodecylamine at the 5′ end of the oligonucleotide. Preferably and in some embodiments, the oligonucleotide is a microRNA. More preferably and in some preferred embodiments, the oligonucleotide consists of a sequence of SEQ ID NO: 1. Preferably and in some embodiments, the oligonucleotide therapeutics further comprise a PEG conjugated to the dodecylamine at the amino terminus of the dodecylamine. Preferably and in some embodiments, the oligonucleotide therapeutics further comprise a peptide linker disposed between the dodecylamine and the PEG. More preferably and in some preferred embodiments, the PEG is selected from the group consisting of PEG 500, PEG 1000, and PEG 2000. Even more preferably and in some more preferred embodiments, the PEG is PEG 500. More preferably and in some preferred embodiments, the peptide linker is conjugated to the dodecylamine at the amino terminus of the dodecylamine, and the PEG is conjugated to the peptide linker at the amino terminus of the peptide linker. More preferably and in some preferred embodiments, the peptide linker consists of a sequence of SEQ ID NO: 2.

The present invention also provides a composition comprising at least one of the oligonucleotide therapeutics of the present invention and a pharmaceutically acceptable carrier or excipient.

The present invention also provides a method of increasing the expression of PD-L1 of a cell, comprising contacting the cell with one of the oligonucleotide therapeutics of the present invention. The present invention further provides a method of increasing the expression of PD-L1 in a subject, comprising administering to the subject one of the oligonucleotide therapeutics of the present invention. The present invention further provides a method of preventing, attenuating, suppressing, or treating inflammation in a subject, comprising administering to the subject a pharmaceutically effective amount of one of the oligonucleotide therapeutics of the present invention.

As used herein, the terms “Programmed Cell Death Protein 1,” “PD-1,” or “CD279 (cluster of differentiation 279)” refer to a cell surface receptor protein found on certain immune cells, particularly T cells, and having a role in regulating the immune system's response to the cells of the human body by down-regulating the immune system and promoting self-tolerance by suppressing T cell inflammatory activity.

As used herein, the terms “Programmed Cell Death Ligand 1,” “PD-L1,” or “CD274 (cluster of differentiation 274)” refer to a 40 kDa type 1 transmembrane protein found on the surface of some cells, including cancer cells and immune cells, and being able to interact with the inhibitory checkpoint molecule PD-1.

As used herein, the term “PD-1/PD-L1 axis” refers to a crucial immune regulatory pathway in the human body via the interaction between PD-1 and PD-L1. The binding of PD-1 on the surface of a T cell to PD-L1 on the surface of another cell transmits an inhibitory signal to reduce the proliferation of antigen-specific T-cells in lymph nodes, while simultaneously reducing apoptosis in regulatory T cells (anti-inflammatory, suppressive T cells). The primary function of this PD-1/PD-L1 interaction is to prevent an overactive immune response that could lead to autoimmunity or excessive tissue damage. However, it is also exploited by certain cancers as a way to evade the immune system. Cancer cells can express PD-L1, and when they engage with PD-1 on T cells, they can effectively dampen the T cell's ability to attack the tumor. This mechanism is one of the ways cancer cells can evade immune surveillance. In addition, PD-1/PD-L1 axis is found as an important pathway for regulating the immune system in the brain, sustaining microglial Aβ uptake and reducing chronic neuroinflammation. Studies have shown that the expression of astrocytic PD-L1 and microglial PD-1 are upregulated around Aβ plaques, and the PD-L1 is secreted in a soluble form by astrocytes to bind to the PD-1 on microglia. The binding of PD-L1 and PD-1 increases microglia's uptake and clearing AB and inhibiting the continuous expansion of AB, thereby inhibiting neuroinflammation (Kummer et al., 2021). Therefore, increased expression of PD-L1 on nerve cells inhibits neuroinflammation.

As used herein, the term “dodecylamine” refers to an organic compound having the formula of C12H27NH2 and the following chemical structure:

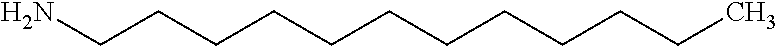

Dodecylamine belongs to the amine class, which is characterized by a primary amine functional group (-NH2) attached to a C12 carbon alkyl chain. As used in the present invention, the first carbon of dodecylamine is conjugated to an oligonucleotide at a 5′ end of the oligonucleotide, and the amino group of the dodecylamine is located at a twelfth carbon of the dodecylamine.

As used herein, the terms “polyethylene glycol” or “PEG” refer to a polymer compound having the formula of H—(O—CH₂—CH₂)ₙ—OH and the following chemical structure:

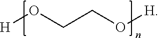

PEG may be followed by a number which represents the average molecular weight. For example, PEG 500, PEG 1000, and PEG 2000 represent PEG whose average molecular mass are 500, 1000, and 2000, respectively.

As used herein, the term “nucleotide” refers to a monomer comprising a nitrogenous base connected to a sugar phosphate that comprises a sugar, such as ribose or 2′-deoxyribose, connected to one or more phosphate groups. “Polynucleotide” and “nucleic acid” refer to a polymer comprising more than one nucleotide monomer, in which said monomers are often connected by sugar-phosphate linkages of a sugar-phosphate backbone. A polynucleotide need not comprise only one type of nucleotide monomer. For example, the nucleotides comprising a given polynucleotide may be only ribonucleotides, only 2′-deoxyribonucleotides, or a combination of both ribonucleotides and 2′-deoxyribonucleotides. Polynucleotides include naturally occurring nucleic acids, such as deoxyribonucleic acid (“DNA”) and ribonucleic acid (“RNA”), as well as nucleic acid analogs comprising one or more non-naturally occurring monomer. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form, but in which “T” replaces “U.” The term “recombinant nucleic acid” refers to a polynucleotide or nucleic acid having sequences that are not naturally joined together. A recombinant nucleic acid may be present in the form of a vector.

As used herein, the terms “oligonucleotide” refers to a short DNA or RNA molecules, which usually has 13-25 nucleotides long. The maximum length of oligonucleotides is around 200 nucleotide residues.

As used herein, the terms “micro ribonucleic acid,” “microRNA,” or “miRNA” refers to a short, non-coding single-stranded RNA sequence consisting of 18-22 nucleotides. MicroRNA binds to the complementary untranslated region (3′-UTR) of messenger RNA (mRNA) to regulate target genes, leading to translational inhibition or degradation of the target genes. Each miRNA can regulate many or even hundreds of different mRNA molecules, and multiple miRNAs can regulate the same mRNA. MicroRNA is involved in various biological functions, including development, differentiation, proliferation, apoptosis.

As used herein, the terms “microRNA 200c-3p” or “miRNA 200c-3p” refer to a specific microRNA molecule belonging to the microRNA-200 family and having the sequence of 5′-UAAUACUGCCGGGUAAUGAUGGA-3′ (SEQ ID NO: 1). Studies have shown that miR-200c-3p can reduce PD-L1, c-Myc, and B-catenin expression in ovarian cancer, suggesting that miR-200c-3p can act as a tumor suppressor in Epithelial ovarian cancer (Anastasiadou et al., 2021). In addition, other studies have shown that miR-200c can inhibit the expression of PD-L1 mRNA in mouse lung tumor cells as an anti-tumor effect (Zhang et al., 2023).

As used herein, the nomenclature used to describe peptides of the invention follows the conventional practice wherein the amino group (N-terminus) and/or the 5′ are presented to the left and the carboxyl group (C-terminus) and/or 3′ are presented to the right.

As used herein, the term “peptide” refers to a molecular chain of amino acids, including both L-forms and D-forms. The amino acids, if required, can be modified in vivo or in vitro, for example by manosylation, glycosylation, amidation (specifically C-terminal amides), carboxylation or phosphorylation with the stipulation that these modifications must preserve the biological activity of the original molecule. In addition, peptides can be part of a chimeric protein.

As used herein, the term “peptide linker” refers to a short chain of amino acids (peptide) used to connect or link different functional components in various biological or chemical molecules.

Functional derivatives of the peptides are also included in the present invention.

Functional derivatives are meant to include peptides which differ in one or more amino acids in the overall sequence, which have deletions, substitutions, inversions or additions. Amino acid substitutions which can be expected not to essentially alter biological and immunological activities have been described. Amino acid replacements between related amino acids or replacements which have occurred frequently in evolution include, inter alia Ser/Ala, Ser/Gly, Asp/Gly, Asp/Asn and Ile/Val.

The peptides according to the invention can be produced synthetically or by recombinant DNA technology. Methods for producing synthetic peptides are well known in the art.

The organic chemical methods for peptide synthesis are considered to include the coupling of the required amino acids by means of a condensation reaction, either in homogenous phase or with the aid of a so-called solid phase. The condensation reaction can be carried out as follows: Condensation of a compound (amino acid, peptide) with a free carboxyl group and protected other reactive groups with a compound (amino acid, peptide) with a free amino group and protected other reactive groups, in the presence of a condensation agent. Condensation of a compound (amino acid, peptide) with an activated carboxyl group and free or protected other reaction groups with a compound (amino acid, peptide) with a free amino group and free or protected other reactive groups. Activation of the carboxyl group can take place, inter alia, by converting the carboxyl group to an acid halide, azide, anhydride, imidazolide or an activated ester, such as the N-hydroxy-succinimide, N-hydroxy-benzotriazole or p-nitrophenyl ester.

As used herein, “pharmaceutically acceptable carrier” or “pharmaceutically acceptable excipient” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal or transdermal administration. Depending on the route of administration, the active compound may be coated in a material to protect the compound from the action of acids and other natural conditions which may inactivate the compound. The use of such media and agents for pharmaceutically active substances is well known in the art.

Formulations suitable for administration of the present invention may comprise, possibly among other things well known to those of skill in the art: aqueous and non-aqueous solutions, antioxidants, bacteriostats, buffers, solutes that affect isotonicity, preservatives, solubilizers, stabilizers, suspending agents, thickening agents, or a combination thereof.

In addition or in the alternative, formulations suitable for administration of the present invention may comprise, possibly among other things well known to those of skill in the art: gels, PEG such as PEG 400, propylene glycol, saline, sachets, water, other appropriate liquids known in the art, or a combination thereof.

Also in the addition or in the alternative, formulations suitable for administration of the present invention may comprise, possibly among other things well known to those of skill in the art: binders, buffering agents, calcium phosphates, cellulose, colloids, such as colloidal silicon dioxide, colorants, diluents, disintegrating agents, dyes, fillers, flavoring agents, gelatin, lactose, magnesium stearate, mannitol, microcrystalline gelatin, moistening agents, paraffin hydrocarbons, pastilles, polyethylene glycols, preservatives, sorbitol, starch, such as corn starch, potato starch, or a combination thereof, stearic acid, sucrose, talc, triglycerides, or a combination thereof.

Also in addition or in the alternative, formulations suitable for administration of the present invention may comprise, possibly among other things well known to those of skill in the art: alcohol such as benzyl alcohol or ethanol, benzalkonium chloride, buffers such as phosphate buffers, acetate buffers, citrate buffers, or a combination thereof, carboxymethylcellulose or microcrystalline cellulose, cholesterol, dextrose, juice such as grapefruit juice, milk, phospholipids such as lecithin, oil such as vegetable, fish, or mineral oil, or a combination thereof, other pharmaceutically compatible carriers known in the art, or a combination thereof.

Also in the addition or in the alternative, formulations suitable for administration of the present invention may comprise, possibly among other things well known to those of skill in the art: biodegradables such as poly-lactic-coglycolic acid (PLGA) polymer, other entities whose degradation products can quickly be cleared from a biological system, or a combination thereof.

Formulations of the present invention may be administered in unit-dose form, multi-dose form, or a combination thereof. They may be packaged in unit-dose containers, multi-dose containers, or a combination thereof. The present invention may exist in ampoules, cachets, capsules, granules, lozenges, powders, tablets, vials, emulsions, including but not limited to acacia emulsions, suspensions, or a combination thereof.

As used herein, an “effective amount” or a “sufficient amount” of a substance is that amount sufficient to effect beneficial or desired results, including clinical results, and, as such, an “effective amount” depends upon the context in which it is being applied. In the context of administering an immunogenic composition, the effective amount is an immunogenically effective amount, which contains sufficient immunogenic composition of the present invention to elicit an immune response. In the context of administering a pharmaceutical composition, the effective amount is a pharmaceutically effective amount, which contains sufficient pharmaceutical composition of the present invention to maintain or produce a desired physiological result. An effective amount can be administered in one or more doses.

As used herein, the term “pharmaceutically effective amount” refers to an amount capable of or sufficient to maintain or produce a desired physiological result, including but not limited to treating, reducing, attenuating, eliminating, suppressing, substantially preventing, or prophylaxing, or a combination thereof, a disease, disorder, or combination thereof. A pharmaceutically effective amount may comprise one or more doses administered sequentially or simultaneously. Those skilled in the art will know to adjust doses of the present invention to account for various types of formulations, including but not limited to slow-release formulation. As used herein, the term “prophylactic” refers to a composition capable of substantially preventing or prophylaxing any aspect of a disease, disorder, or combination thereof. As used herein, the term “therapeutic” refers to a composition capable of treating, reducing, halting the progression of, slowing the progression of, beneficially altering, eliminating, or a combination thereof, any aspect of a disease, disorder, or combination thereof.

The term “dose” as used herein in reference to a composition refers to a measured portion of the composition taken by (administered to or received by) a subject at any one time.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In some embodiments, the subject is a human. Human subjects may also include fetuses.

As used herein, the terms “subject,” refers to any subject, particularly a mammalian subject, for whom therapy is desired, for example, a human.

The term “treat,” “treating,” or “treatment” as used herein encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevent,” “preventing,” or “prevention” refers to being able to substantially preclude, avert, obviate, forestall, stop, hinder, or a combination thereof, any aspect of a disease, condition, or combination thereof from happening, especially by advance action.

In some embodiments, the oligonucleotide therapeutics and/or the composition of the present invention may be administered to subjects by a variety of administration modes, including by intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes. Alternatively, the antigen may be administered ex-vivo by direct exposure to cells, tissues or organs originating from a subject (autologous) or another subject (allogeneic), optionally in a biologically suitable, liquid or solid carrier.

The meaning of the technical and scientific terms as described herein can be clearly understood by a person of ordinary skill in the art.

As used herein, the term “about,” “around,” or “approximately” when combined with a value refers to plus and minus 10% of the reference value. For example, a length of about 1000 nanometers (nm) refers to a length of the range of 900 nm to 1100 nm.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1 Preparation of the Oligonucleotide Therapeutics

### Materials and Methods

Design and Preparation of Oligonucleotide Therapeutics. The schematic

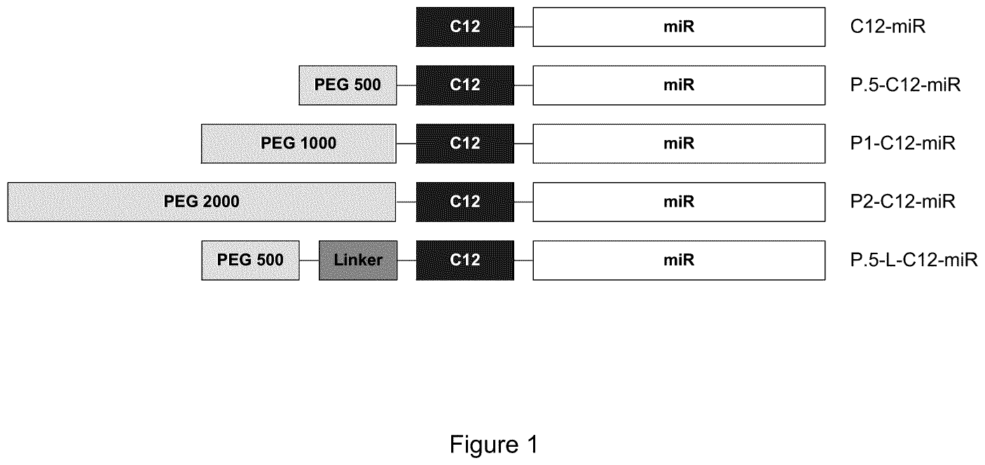

diagrams of the design of the oligonucleotide therapeutics disclosed in the Examples are shown in FIG. 1. Preparation of the oligonucleotide therapeutics is described below. First of all, microRNA 200c-3p (5′-UAAUACUGCCGGGUAAUGAUGGA-3′; SEQ ID NO: 1) was synthesized with solid phase synthesis (Genomics, New Taipei City, Taiwan; GenScript Biotech, NJ, US) and modified with a dodecylamine (C₁₂H₂₇N) at the 5′ end of the microRNA, in which the first carbon of the dodecylamine was conjugated to the microRNA at its 5′ end and the amino group of the dodecylamine was located at the twelfth carbon of the dodecylamine. The obtained oligonucleotide therapeutic was designated as C12-miR.

The dodecylamine modified microRNA 200c-3p (SEQ ID NO: 1) (C12-miR) was further conjugated to different sizes (0.5, 1 and 2 kDa) of polyethylene glycol (PEG) at the amino terminus of the dodecylamine. Pegylation of C12-miR was performed as follows: 4.15 nM C12-miR and 4.15 nM PEG (0.5, 1, or 2 kDa) were mixed in MES (2(N-morpholino)ethanesulfonic acid) buffer containing 4.15 nM EDC (1-(3-Dimethylaminopropyl)-3-ethylcarbodiimide hydrochloride) at room temperature for 3 hours. The mixture was then purified with Microspin™ G-25 column (Sigma-Aldrich, MO, US) to obtain more oligonucleotide therapeutics of this Example. The C12-miRs modified with PEG 500 (0.5 kDa), PEG 1000 (1 kDa), and PEG 2000 (2 kDa) were designated as P.5-C12-miR, P1-C12-miR, and P2-C12-miR, respectively.

In addition, the dodecylamine modified microRNA 200c-3p (SEQ ID NO: 1) (C12-miR) was further modified with a peptide linker (SEQ ID NO: 2) at the amino terminus of the dodecylamine, and the peptide linker (KGDGG; SEQ ID NO: 2) was further conjugated to PEG 500 (0.5 kDa) at the amino terminus of the peptide linker to form another oligonucleotide therapeutic, designated as P.5-L-C12-miR. In brief, 4.15 nM C12-miR, 4.15 nM peptide linker (SEQ ID NO: 2), and 4.15 nM PEG 500 were mixed in MES buffer containing 4.15 nM EDC at room temperature for 3 hours. The mixture was then purified with Microspin™ G-25 column to obtain the oligonucleotide therapeutic P.5-L-C12-miR.

The amount of pegylated C12-miR products (P.5-C12-miR, P1-C12-miR, P2-C12-miR, and P.5-L-C12-miR) was measured with an optical density at 260 nm of wavelength by NanoDrop One Spectrophotomete (Thermo Fisher Scientific, MA, USA). The unpegylated and pegylated C12-miRs (C12-miR, P.5-C12-miR, P1-C12-miR, P2-C12-miR, and P.5-L-C12-miR) were characterized by electrophoresis in a 15% (v/v) acrylamide gel containing 8 M urea followed by staining with 0.2% (w/v) methylene blue for 20-30 minutes.

### Results

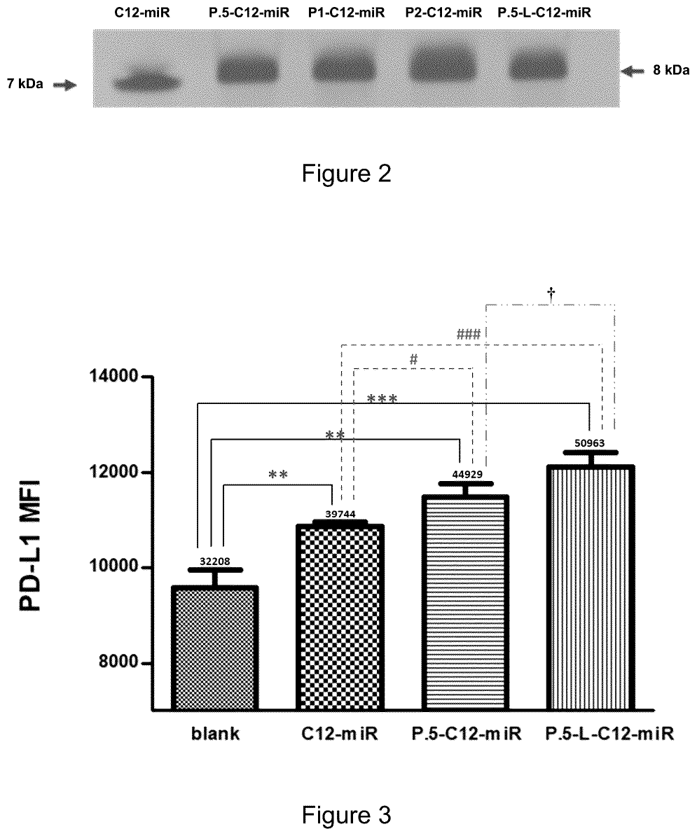

As shown in FIG. 2, the unpegylated C12-miR has an expected size of around 7 kDa. As the sizes of the conjugated PEGs increase, the sizes of the pegylated C12-miRs (P.5-C12-miR, P1-C12-miR, and P2-C12-miR) also increase. The pegylated peptide-linked C12-miR (P.5-L-C12-miR) has a bigger size than P.5-C12-miR due to the addition of the peptide linker (SEQ ID NO: 2).

### Example 2 Biological Function Assay of Pegylated Oligonucleotide

### Therapeutics Modified With or Without a Peptide Linker

### Materials and Methods

Cell treatment. SH-SY5Y human neuroblastoma cells (ATCC® CRL-2266) suspended in MEM/F12K medium (1:1, v/v) were seeded onto a 96-well microplate at a density of around 5×10³ cells/well. The cells were incubated at 37° C., 5% CO₂ for 16 hours before treated with 2 μM of C12-miR, P.5-C12-miR, and P.5-L-C12-miR obtained in Example 1, respectively. The treated cells were then incubated at 37° C., 5% CO₂ for 48 more hours. Cells treated with phosphate-buffered saline (PBS) were used as blank control.

Flow cytometry. Cells were harvested and washed with PBS. The collected cells were stained with anti-human PD-L1 surface antibodies (Cat. No. 329706, Biolegend, CA, US) and incubated in the dark at 4° C. for 30 minutes. The cells were then washed twice in cold FACS buffer, resuspended in FACS buffer, and analyzed in a flow cytometer (BD LSRFortessa™ X20, NJ, US).

Statistical analysis. All data were compiled by Student's 1-test (TTEST, Excel, Microsoft, WA, US) for one-tail test. P value <0.05 between groups was considered as significant difference.

### Results

The oligonucleotide therapeutics of the present invention induce PD-L1 expression. As shown in FIG. 3, the three tested oligonucleotide therapeutics, C12-miR, P.5-C12-miR, and P.5-L-C12-miR, significantly increase the PD-L1 expression of SH-SY5Y cells by 23% (p<0.01), 39% (p<0.01), and 58% (p<0.001), respectively, as compared with the blank control. In particular, the pegylated C12-miR (P.5-C12-miR) (p<0.05) and the pegylated peptide-linked C12-miR (P.5-L-C12-miR) (p<0.001) significantly increase the PD-L1 expression of SH-SY5Y cells as compared with the unpegylated C12-miR. More specifically, the pegylated peptide-linked C12-miR (P.5-L-C12-miR) also significantly increase the PD-L1 expression of SH-SY5Y cells as compared with the pegylated C12-miR (P.5-C12-miR) (p<0.05).

The results indicate that the dodecylamine modified microRNA 200c-3p (SEQ ID NO: 1) (C12-miR) surprisingly increases PD-L1 expression of cells, which is completely opposite to the downregulating effects of microRNA 200c-3p mimic or microRNA 200c-3p expression vector on PD-L1 expression of cells as previously reported (Anastasiadou et al., 2021; Zhang et al., 2023). The results also suggest that pegylation of the dodecylamine modified microRNA 200c-3p (SEQ ID NO: 1) (P.5-C12-miR) induces more PD-L1 expression of cells than the dodecylamine modified microRNA 200c-3p (SEQ ID NO: 1) (C12-miR). The results further suggest that the addition of a peptide linker (SEQ ID NO: 2) between the PEG and the dodecylamine modified microRNA 200c-3p (SEQ ID NO: 1) (P.5-L-C12-miR) induces even more PD-L1 expression of cells than the dodecylamine modified microRNA 200c-3p (SEQ ID NO: 1) (C12-miR).

### Example 3 Biological Function Assay of Pegylated Oligonucleotide Therapeutics With Different Dose

### Materials and Methods

Cell treatment. SH-SY5Y human neuroblastoma cells (ATCC® CRL-2266) were also used in this Example. Cells were cultured as described in Example 2, except being treated with 0.5 μM, 2 μM, and 4 μM of C12-miR or P.5-C12-miR in this Example. Cells treated with PBS were used as blank control (i.e., 0 μM of C12-miR or P.5-C12-miR).

Flow cytometry. Method of flow cytometry are the same as described in Example 2.

Statistical analysis. Method of statistical analysis are the same as described in Example 2.

### Results

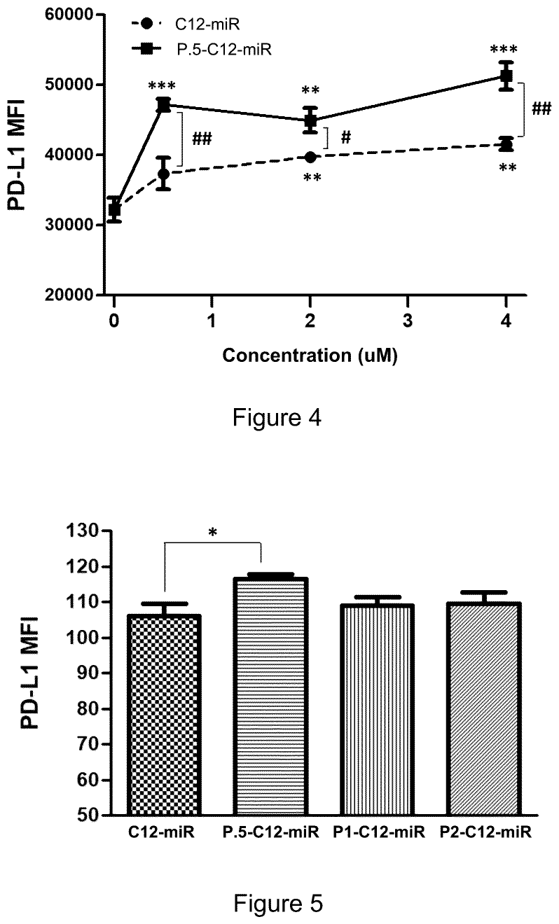

The oligonucleotide therapeutics of the present invention induce PD-LI expression in a dose-dependent manner. As shown in FIG. 4, 0.5 μM, 2 μM, and 4 μM of the dodecylamine modified microRNA 200c-3p (SEQ ID NO: 1) (C12-miR) significantly increases the PD-L1 expression of SH-SY5Y cells by 16%, 23% (p<0.01), and 29% (p<0.01), respectively, as compared with the blank control (0 μM). Similarly, 0.5 μM, 2 μM, and 4 μM of the pegylated C12-miR (P.5-C12-miR) significantly increases the PD-L1 expression of SH-SY5Y cells by 46% (p<0.001), 39% (p<0.001), and 59% (p<0.001), respectively, as compared with the blank control (0 μM). In addition, 0.5 μM, 2 μM, and 4 μM of P.5-C12-miR significantly induces more PD-L1 expression of SH-SY5Y cells than 0.5 μM, 2 μM, and 4 μM of C12-miR, respectively (p<0.05 or p<0.01), indicating that pegylation of C12-miR (P.5-C12-miR) has better effects on inducing PD-L1 expression of cells than C12-miR. The results suggest that both C12-miR and peglated C12-miR (P.5-C12-miR) increase PD-L1 expression of neuroblastoma cells in a dose-dependent manner.

### Example 4 Biological Function Assay of Oligonucleotide Therapeutics Pegylated With Different Sizes of PEG

### Materials and Methods

Cell treatment. SH-SY5Y human neuroblastoma cells (ATCC® CRL-2266) were also used in this Example. Cells were cultured as described in Example 2, except being treated with 2 μM of C12-miR, P.5-C12-miR, P1-C12-miR, or P2-C12-miR in this Example. Cells treated with 2 μM of C12-miR were used as positive control.

Flow cytometry. Method of flow cytometry are the same as described in Example 2.

Statistical analysis. Method of statistical analysis are the same as described in Example 2.

### Results

The oligonucleotide therapeutics pegylated with different sizes of PEG all induce PD-LI expression. As shown in FIG. 5, 2 μM of P.5-C12-miR, P1-C12-miR, and P2-C12-miR increase the PD-L1 expression of SH-SY5Y cells by 10% (p<0.05), 2%, and 3%, respectively, as compared with 2 μM of C12-miR. Among these pegylated C12-miR, the C12-miR pegylated with PEG 500 (P.5-C12-miR) significantly induces the most PD-L1 expression of cells (p<0.05). The results suggest that pegylation of C12-miR with different sizes of PEG has a positive impact on inducing PD-L1 expression of cells, especially pegylation with PEG 500.

### Example 5 Cleavage Assay of the Peptide Linker

### Materials and Methods

Cleavage Assay. The pegylated peptide-linked C12-miR (P.5-L-C12-miR) obtained in Example 1 was subjected to enzyme cleavage by lysosomal enzyme Cathepsin D in vitro. In brief, 10 μL of P.5-L-C12-miR (4.15 nM) and 2 μL Cathepsin D (Cat. No. C8696, Sigma-Aldrich, MO, USA) were added into 250 mM sodium acetate solution (pH 3.7) to a final volume of 20 μL. The reaction mixture was incubated at 37° ° C. for 5 hours. The reaction resultant was analyzed by electrophoresis in a 15% (v/v) acrylamide gel containing 8 M urea with 200 voltages for 1 hour. The gel was then stained with 0.2% (w/v) methylene blue for 20-30 minutes and imaged by Gel Doc EZ (Bio-Rad, CA, USA) integrated with an image analysis software (Image Lab, Bio-Rad, CA, USA). Each band stained for the nucleic acid part was analyzed, and the peak density of each band was taken as the size of that band.

### Results

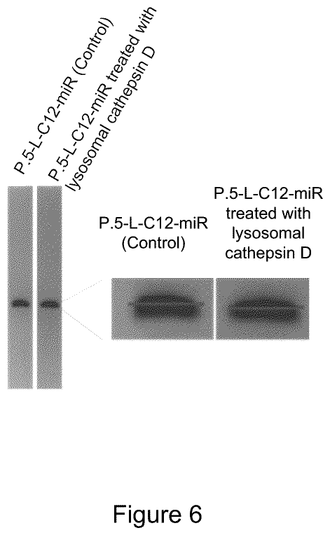

The peptide linker can be cleaved by cathepsin D. As shown in FIG. 6, the pegylated peptide-linked C12-miR (P.5-L-C12-miR) was cleaved by lysosomal cathepsin D and resulted in a smaller fragment. The result indicates that after the oligonucleotide therapeutics P.5-L-C12-miR is taken by a cell via pinocytosis/phagocytosis, the dodecylamine modified microRNA 200c-3p (SEQ ID NO: 1) (C12-miR) can be released into the cell through enzymatic cleavage of the peptide linker by lysosomal enzymes to increase the effect of C12-miR on the cell.

In conclusion, the dodecylamine modified microRNA 200c-3p (SEQ ID NO: 1) (C12-miR) surprisingly increases PD-L1 expression of cells, and pegylation of C12-miR induces more PD-L1 expression. The addition of the peptide linker (SEQ ID NO: 2) between the PEG and C12-miR induces even more PD-L1 expression of cells. In addition, the enzymatic cleavage of the peptide linker (SEQ ID NO: 2) allows the dodecylamine modified microRNA 200c-3p (SEQ ID NO: 1) (C12-miR) be further released into the cells. The results suggest that the oligonucleotide therapeutics of the present invention can be used to prevent, attenuate, suppress, and treat inflammatory responses, especially neuroinflammation.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

### References

1. G. S. Bloom, Amyloid-β and Tau. The trigger and bullet in Alzheimer disease pathogenesis. JAMA Neurology. 2014; 71: 505-508; doi: 10.1001/jamaneurol.2013.5847.
2. J. W. Kinney et al., Inflammation as a central mechanism in Alzheimer's disease. Alzheimers Dement (NY). 2018; 4: 575-590; doi: 10.1016/j.trci.2018.06.014.
3. G. Lueg. et al., Clinical relevance of specific T-cell activation in the blood and cerebrospinal fluid of patients with mild Alzheimer's disease. Neurobiol. Aging. 2015; 36: 81-89; doi: 10.1016/j.neurobiolaging.2014.08.008.
4. D. V. Hansen et al., Microglia in Alzheimer's disease. J. Cell. Biol. 2018; 217: 459-472; doi: 10.1083/jcb.201709069.
5. E. Anastasiadou et al., MiR-200c-3p contrasts PD-L1 induction by combinatorial therapies and slows proliferation of epithelial ovarian cancer through downregulation of B-catenin and c-Myc. Cells 2021; 10: 519; doi: 10.3390/cells10030519.
6. Q. Zhang et al., Aerosolized miR-138-5p and miR-200c targets PD-L1 for lung cancer prevention. Front. Immunol., 2023; 14:1166951; doi: 10.3389/fimmu.2023.1166951.
7. Kummer et al., Microglial PD-1 stimulation by astrocytic PD-L1 suppresses neuroinflammation and Alzheimer's desease pathology. EMBO J. 2021; 40:e108662; doi: 10.15252/embj.2021108662.

What is claimed is:

1. An oligonucleotide therapeutic, comprising an oligonucleotide and a dodecylamine, wherein a first carbon of the dodecylamine is conjugated to the oligonucleotide at a 5′ end of the oligonucleotide, and an amino group of the dodecylamine is located at a twelfth carbon of the dodecylamine.

2. The oligonucleotide therapeutic of claim 1, wherein the oligonucleotide is a microRNA.

3. The oligonucleotide therapeutic of claim 1, wherein the oligonucleotide consists of a sequence of SEQ ID NO: 1.

4. The oligonucleotide therapeutic of claim 1, further comprising a polyethylene glycol (PEG) conjugated to the dodecylamine at an amino terminus of the dodecylamine.

5. The oligonucleotide therapeutic of claim 4, wherein the PEG is selected from the group consisting of PEG 500, PEG 1000, and PEG 2000.

6. The oligonucleotide therapeutic of claim 1, further comprising a peptide linker conjugated to the dodecylamine at an amino terminus of the dodecylamine amino terminus of the dodecylamine and a PEG conjugated to the peptide linker at an amino terminus of the peptide linker.

7. The oligonucleotide therapeutic of claim 6, wherein the peptide linker consists of a sequence of SEQ ID NO: 2.

8. The oligonucleotide therapeutic of claim 6, wherein the PEG is selected from the group consisting of PEG 500, PEG 1000, and PEG 2000.

9. The oligonucleotide therapeutic of claim 1, wherein the oligonucleotide therapeutic is selected from the group consisting of
an oligonucleotide therapeutic consisting of an oligonucleotide and a dodecylamine, wherein a first carbon of the dodecylamine is conjugated to the oligonucleotide at a 5′ end of the oligonucleotide, and an amino group of the dodecylamine is located at a twelfth carbon of the dodecylamine;
an oligonucleotide therapeutic consisting of an oligonucleotide, a dodecylamine, and a PEG, wherein a first carbon of the dodecylamine is conjugated to the oligonucleotide at a 5′ end of the oligonucleotide, an amino group of the dodecylamine is located at a twelfth carbon of the dodecylamine, and the PEG is conjugated to the dodecylamine at an amino terminus of the dodecylamine; and
an oligonucleotide therapeutic consisting of an oligonucleotide, a dodecylamine, a peptide linker, and a PEG, wherein a first carbon of the dodecylamine is conjugated to the oligonucleotide at a 5′ end of the oligonucleotide, an amino group of the dodecylamine is located at a twelfth carbon of the dodecylamine, the peptide linker is conjugated to the dodecylamine at an amino terminus of the dodecylamine amino terminus of the dodecylamine, and the PEG is conjugated to the peptide linker at an amino terminus of the peptide linker.

10. The oligonucleotide therapeutic of claim 9, wherein the oligonucleotide is a microRNA.

11. The oligonucleotide therapeutic of claim 9, wherein the oligonucleotide consists of a sequence of SEQ ID NO: 1.

12. The oligonucleotide therapeutic of claim 9, wherein the PEG is selected from the group consisting of PEG 500, PEG 1000, and PEG 2000.

13. The oligonucleotide therapeutic of claim 9, wherein the peptide linker consists of a sequence of SEQ ID NO: 2.

14. A composition, comprising the oligonucleotide therapeutic of claim 1 and a pharmaceutically acceptable carrier or excipient.

15. A method of increasing the expression of PD-L1 of a cell, comprising contacting the cell with the oligonucleotide therapeutic of claim 1.

16. A method of increasing the expression of PD-L1 of a cell, comprising contacting the cell with the composition of claim 14.

17. A method of increasing the expression of PD-L1 in a subject, comprising administering to the subject the oligonucleotide therapeutic of claim 1.

18. A method of increasing the expression of PD-L1 in a subject, comprising administering to the subject the composition of claim 14.

19. A method of attenuating inflammation in a subject, comprising administering to the subject a pharmaceutically effective amount of the oligonucleotide therapeutic of claim 1.

20. A method of attenuating inflammation in a subject, comprising administering to the subject a pharmaceutically effective amount of the composition of claim 14.
